(19) United States
(12) Patent Application Publication
(10) Pub. No.: US 2023/0181177 A1
(43) Pub. Date: Jun. 15, 2023

(54) Micro Punch for Targeted Material Excision

(71) Applicants: University of Massachusetts, Boston, MA (US); Worcester Polytechnic Institute, Worcester, MA (US)

(72) Inventors: David M. Harlan, Northborough, MA (US); Yihao Zheng, Westwood, MA (US); Sambra D. Redick, Spencer, MA (US); Katerina Angjeli, Belmont, MA (US); Sally C. Kent, Worcester, MA (US)

(21) Appl. No.: 18/065,549

(22) Filed: Dec. 13, 2022

Related U.S. Application Data
(60) Provisional application No. 63/289,196, filed on Dec. 14, 2021.

(51) Int. Cl. A61B 10/02; B23B 31/20

(52) U.S. Cl. CPC A61B 10/0266; B23B 31/20

(57) ABSTRACT

Described are a micro punch cutting tool and associated method that can be used under a microscope to accurately excise a targeted piece of material on a micron or millimeter scale. An example device for targeted material excision from a tissue sample includes a support defining an opening and providing a region below the opening for positioning a tissue sample. A tissue stabilizer is receivable in the opening and defines a shape that complements a shape of the support. A cutting tool assembly receivable in the tissue stabilizer includes a hollow cutter having a bore and is movable relative to the tissue stabilizer to cause the cutter to excise material from the tissue sample when the tissue sample is positioned below the opening of the support. Movement of the cutting tool assembly is guided by a helical groove in the tissue stabilizer engaging a protrusion of the cutting tool assembly.

### RELATED APPLICATION

This application claims the benefit of U.S. Provisional Application No. 63/289,196, filed on Dec. 14, 2021.

The entire teachings of the above application are incorporated herein by reference.

### BACKGROUND

Punching, e.g., excising, a region of interest from a tissue slice with a hand-held needle-shaped cutter or punch tool has been described in the literature. Hand holding the punch tool typically obstructs the view of the tissue slice on the microscope, making it difficult to simultaneously visualize both the region of interest and the location of the punch. In addition, the previously described approaches either used thick (˜3 mm) tissue sections or they employed a mesh to hold down thinner (30-400 μm) sections for punching. Given that thick tissue sections are often fragile, sticky, and largely translucent, it is difficult to be certain that recovery of the punched tissue is complete. Many live tissue sections are cut with a vibrating microtome and range in thickness from 150-300 μm. These tissue sections typically require the use of a hold-down. The use of a mesh hold-down limits the regions of tissue that are available for recovery.

### SUMMARY

A device, e.g., a micro punch cutting tool, and associated method are described that can be used under a microscope to accurately excise a targeted piece of material, e.g., material from live or fixed tissue, on a micron or millimeter scale.

An example device for targeted material excision from a tissue sample includes a support defining an opening and providing a region below the opening for positioning a tissue sample. A tissue stabilizer is receivable in the opening of the support, the tissue stabilizer defining a shape that complements a shape of the support defining the opening. A cutting tool assembly is receivable in the tissue stabilizer. The cutting tool assembly includes a cutter with an inner bore and being movable relative to the tissue stabilizer to cause the cutter to excise material from the tissue sample when the tissue sample is positioned below the opening of the support.

The opening in the support can be asymmetric, e.g., a circular hole with a vertical groove, and the tissue stabilizer can be asymmetric, e.g., defining an asymmetric shape that complements the asymmetric opening. For example, the tissue stabilizer can include a rib that fits into the vertical groove at the opening of the support.

The tissue stabilizer can include an internal helical groove, e.g., internal threads, and the cutting tool assembly can include, on its outer surface, a protrusion to engage with the internal groove, e.g., the internal threads, of the tissue stabilizer. Movement of the cutting tool assembly relative to the tissue stabilizer can be guided by the protrusion traversing the internal helical groove. In this way, the cutting tool assembly can move in a combined turning and advancing motion to cause the cutter to excise material from the tissue sample and retain tissue material within the bore of the cutter. The tissue stabilizer and the cutting tool assembly can be separable parts. The tissue stabilizer and the cutting tool assembly may form one integrated module.

The cutting tool assembly can include a collet chuck configured to hold the cutter in an orientation with an excise end of the cutter directed toward the tissue sample, and can further include a chuck holder to couple to the collet chuck to facilitate manipulation of the cutting tool assembly by a user.

The cutter at one end can include one or more cutting edges to facilitate material separation. The cutter may define an inner cavity or hole to collect the material being separated. The cutter can be of suitable size (ranging from micrometer to millimeter) and geometry (circular or noncircular). The cutter can be made of some suitable base materials, including but not limited to, steel with various coatings, including but not limited to, titanium nitride and diamond.

The support of the device can include a base and a support arm that defines the asymmetric opening of the support. The support arm can be attached, e.g., screwed or otherwise fastened, to the base. The support base can be crescent shaped and configured to at least partially surround the tissue sample when the tissue sample is positioned below the opening of the support.

The device can further include a plunger receivable in the bore of the cutter to expel material excised from the tissue sample.

The device can be provided in combination with a substrate, e.g., a punching substrate, which can enable holding of the tissue sample between layers of the substrate. The layers of the substrate can include one or more sheets of transparent film (e.g., plastic film and/or cellophane), a porous layer (e.g., polyacrylamide under-pad), and a rubber cushion. The porous layer can be saturated with nutrient medium.

An example method for targeted excision of material from a tissue sample includes positioning a tissue sample below an opening in a support and inserting a cutting tool assembly into a tissue stabilizer. The cutting tool assembly includes a hollow cutter. The method further includes guiding the tissue stabilizer and cutting tool assembly into the opening in the support, and, while stabilizing the tissue sample, executing a combined turning and advancing movement of the cutting tool assembly relative to the tissue stabilizer and the tissue sample to cause the hollow cutter to excise material from the tissue sample.

The opening in the support can be asymmetric and the tissue stabilizer can define a shape that mirrors the asymmetry of the support. The cutting tool assembly can be provided with an external protrusion that is receivable in an internally-threaded portion of the tissue stabilizer. Moving the cutting tool assembly can include executing a combined turning and advancing movement of the cutting tool assembly relative to the tissue stabilizer and the tissue to cause the cutter to excise material from the tissue sample.

The method can include holding the tissue sample between layers of a substrate including a sheet of transparent film, a porous, nutrient-containing layer, and a rubber cushion. Stabilizing the tissue sample can include contacting the substrate with the tissue stabilizer, securing the tissue sample between the transparent film and the nutrient containing layer with a stabilizing ring, or both. A plunger can be inserted into a bore of the cutter to expel material excised from the tissue sample. Prior to inserting the plunger into the bore of the hollow cutter, the cutting tool assembly can be removed from the tissue stabilizer.

Embodiments of the present invention have several advantages and offer improvements over known techniques and processes. One current process for removing material involves a person physically holding a knife and manually cutting around a small diameter of choice from the tissue. This is not only slow and inefficient, but inaccurate and largely uncontrollable to statistically significant criteria. The handheld method is prone to user error. For example, the user can easily damage the already small and fragile tissue piece, or cut too much or too little to be of any use. Other known approaches are punching regions of interest from live tissue with hand-held needles or punch tools. The present approach uses mechanical support and guidance, and typically requires only an initial calibration that is confirmed by microscopic viewing and thus offers a better way of extracting tissue than the current, handheld methods. The device and method described herein allow for live or fixed samples of a variety of sizes to be placed under the microscope, and for a faster processing time per sample and greater flexibility in terms of sample type and positioning. A faster setup speed is essential in certain applications, for example, when dealing with live tissue samples, and speed is essential when collecting multiple samples from live tissue. The size, placement, and clamping of the excising cutter, and workpiece, can be arranged in such a way that possibility for tissue damage during excision is minimized. This allows for the near elimination of error from the final sample tissue, which in turn removes error from the final research results of the experiments being done with the excised tissue material.

Vibratome and Microtome devices are known technologies to cut thin slices from live or fixed tissue. The Vibratome device cuts thin horizontal slices using vibration, and by adjusting the amplitude and different speeds of the blade. The slices cut from a Vibratome device are thicker than those of a Microtome device, and both products require the tissue to be embedded in a support medium. The support medium most commonly used with a Vibratome is agarose, a gelatin-like substance. Both products are generally not able to aim and make excisions from small tissue. While such products are useful to make thin horizontal cuts, a device according to the present approach is able to punch and extract certain areas of interest from the live or fixed tissue material that has been cut into slices with these devices. This enables looking at the behavior of just a small tissue area, as opposed to the whole tissue slice, which is useful for research purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is an exploded perspective view of a device for targeted material excision illustrating individual parts of the device and how they are assembled according to an example embodiment.

FIG. 2 is an exploded side view of the device of FIG. 1 illustrating how parts of the device are assembled.

FIGS. 3A-3B illustrate details of an example tissue stabilizer, such as the tissue stabilizer of the device of FIG. 1. FIG. 3A is a side view illustrating an example asymmetric external protrusion on the tissue stabilizer. FIG. 3B is a cut away view of FIG. 3A illustrating an example internal helical grove of the tissue stabilizer.

FIGS. 4A-4D illustrate parts used in the recovery of tissue from the cutter of the device according to an example embodiment. FIG. 4A illustrates a collet chuck and needle-shaped cutter assembly and a plunger for recovering tissue. FIG. 4B illustrates recovery of tissue from the collet chuck and cutting tool assembly using the plunger. FIGS. 4C and 4D illustrate a silicone adapter that is designed to make a tight seal with both the needle-shaped cutter and a standard disposable pipet tip, for use in rinsing an internal bore of the cutter with culture medium.

FIGS. 5A-5D are schematic diagrams illustrating tissue before and after punching, and recovered material targeted by the punch. FIG. 5A is a top view of a tissue slice supported by a polyacrylamide under-pad on a silicone support and covered by a transparent film, which is secured with a stabilizer ring. FIG. 5B is an exploded side view of components shown in FIG. 5A. FIG. 5C is a top view of tissue slice after punching. FIG. 5D is a top view of material recovered from the punch into a culture plate with culture medium.

FIG. 6 is an image of mouse live pancreas tissue immobilized by nylon mesh against a silicone under-pad. The red arrowhead indicates an area removed by a device for targeted material excision according to an example embodiment.

FIG. 7 is an image of two pieces of punched live tissue (indicated by white arrows) recovered to a droplet of culture medium. The white circle is a reflection of the light source used for photography.

FIG. 8 is another view of the two recovered live tissue sections (white arrows). In this view, the recovered silicone under-pad pieces are visible (blue arrows).

FIG. 9 is an image of live human pancreas immobilized under cellophane against an under-pad of acrylamide with a silicone support. The red circle indicates the area targeted for removal with a device according to an example embodiment.

FIG. 10 is an image of the live human pancreas tissue shown in FIG. 9 following removal of targeted tissue. The small circle at the bottom of the image is an air bubble that was inadvertently introduced with the punching.

FIG. 11 is a high magnification image of the punched live tissue recovered from the area shown in FIG. 9 and FIG. 10.

### DETAILED DESCRIPTION

A description of example embodiments follows.

Described herein is a device for excising material, also referred to herein as a micro punch or micropunch or micro punch cutting tool, that can be used with a microscope (e.g., a dissecting microscope) to accurately aim and excise a targeted piece of material on a micron or millimeter scale. A method for the use of the device is also described. Preferably, all parts of the device can be sterilized, allowing recovery and culture of targeted regions of living tissue, such as live pancreatic tissue.

The device described herein provides a tool designed to extract a certain material sample with a micron to millimeter scale diameter from a larger slice of live or fixed material and a method for its use to recover the punched material, for example, for the purposes of further research-related inspection. This tool and method of use can address the precision and time-efficiency issues associated with the current methods of such material extraction.

Example features and uses are: to fabricate a punch with a round, sharp, cutting edge of a diameter on microns to millimeters scale; to facilitate targeting and executing the punch in an easy, accurate and mechanized way; to retrieve the excised material; to allow efficient sterilization of the parts of the punch tool that are in contact with both the material to be punched and the excised material.

The present disclosure provides a system that includes a holding device to support thin tissue sections during punching. The system includes a taper point, sharp cutting edge, which is able to make an accurate excision on sensitive materials like live tissue. The size and diameter of the excised material can range from micron to millimeter scale. The excision can be performed under a dissecting microscope. After the punch is made, e.g., material is excised, the punched (e.g., excised) material can be removed and expelled into a culture plate for culture and further examination.

One example of a specific research area that can utilize this device is as follows. The pancreas contains hormone-producing cell clusters called islets. Each islet contains several cell types, including insulin-producing beta cells. In type 1 diabetes (T1D), the pancreas loses the ability to produce insulin because invading immune cells attack and destroy the beta cells. Islets under immune attack can be microscopically visualized in live pancreas sections, then targeted and isolated for further study by utilizing the device and method described herein.

Prior approaches include descriptions of manually-guided hollow needles used to recover tissue samples (see, e.g., Jacobowitz, D. M. Removal of discrete fresh regions of the rat brain, Brain Research 1974, 80: 111-115; Palkovits, M. and M. J. Brownstein. Micro-dissection of brain areas by the punch technique, p. 1-36. In A. C. Cuello (Ed.), Brain Microdissection Techniques. 1983, Wiley, N.Y.). Some of these works describe nylon mesh used in concert with a metal ring to hold down the tissue sections while punching (see, e.g., Johnson, B. P. et al. Vital ex vivo tissue labeling and pathology guided micropunching to characterize cellular heterogeneity in the tissue microenvironment, Biotechniques 2018, 64(1): 13-19). These approaches are sufficient when the punched sample need only be from a region of a relatively solid tissue of interest. However, when the regions of interest are randomly distributed within the tissue as islets within the pancreas are, the likelihood of a region of interest not being at least partially obscured by the grid of the mesh is quite low. In addition, depending on the opacity of the tissue being sampled, manual targeting's accuracy may be compromised by an inability to clearly visualize the sampling needle. Finally, the manual dexterity of the user might not allow the precise targeting that is required to recover regions of interest with a minimum of surrounding tissue. Additionally, because of its fragile gelatinous nature, pancreas tissue requires some method of immobilization and support to prevent the tissue from adhering to the outside of the punching needle in order to allow a clean punch that does not damage the tissue surrounding the area of interest.

In order to address shortcomings of the prior approaches, a device was developed that incorporates a tissue stabilizer. This tissue stabilizer works in conjunction with one or more layers of transparent film (e.g., plastic film and/or cellophane) and a nutrient-filled porous layer (e.g., polyacrylamide) to immobilize and support the tissue. A method of using the device for targeted tissue excision is further described below. Use of the device according to the disclosed method can ensure that removal of the cutter from the tissue following a punch does not distort the tissue. In addition, the device allows precise targeting of the region to be punched, permitting the use of a sufficiently fine cutter to minimize the amount of surrounding tissue captured with the region of interest.

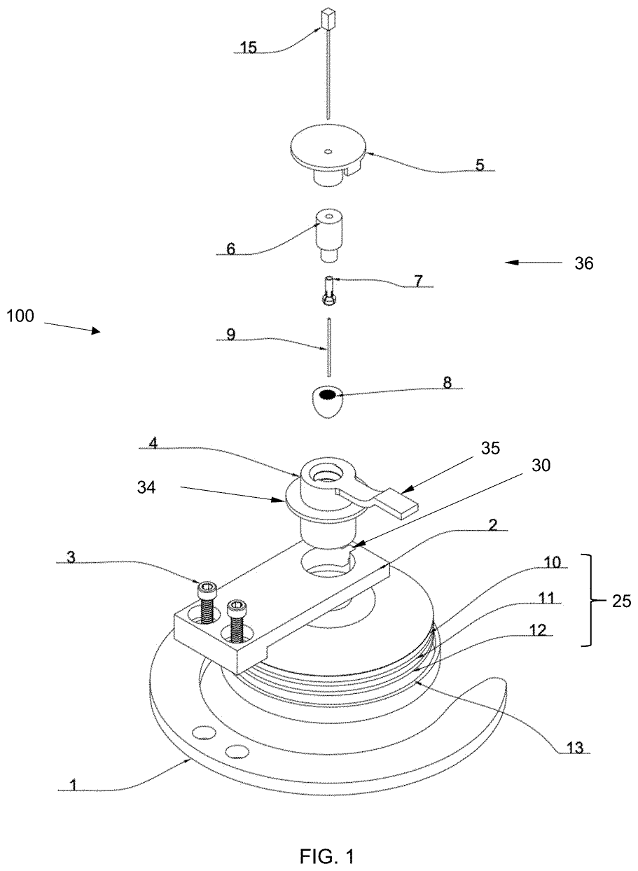

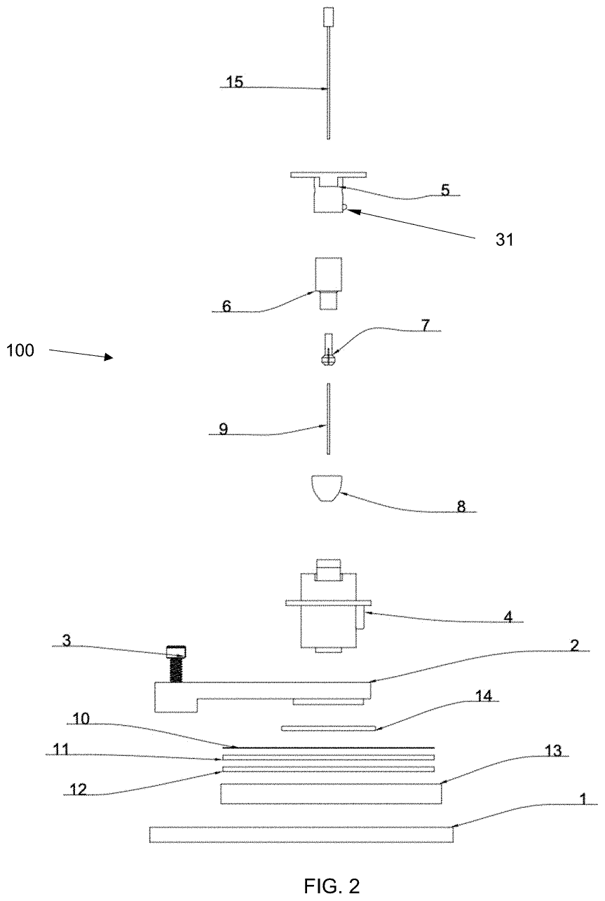

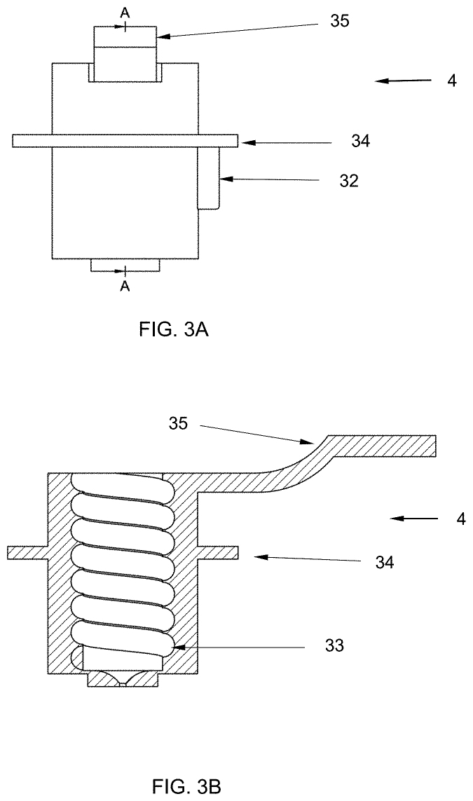

FIGS. 1 and 2 illustrate a device (100) for targeted material excision from a live or fixed tissue sample. The device (100) includes a support that defines an opening (30), e.g., an asymmetric opening, such as a circular hole with a single vertical groove, and provides a region below the opening for positioning a tissue sample. The device further includes a tissue stabilizer (4) that is receivable in the asymmetric opening of the support. A protruding rib (32) of the tissue stabilizer (as illustrated in FIG. 3A) engages with the groove of the support. The tissue stabilizer defines a shape that complements a shape of the support defining the opening. The device further includes a cutting tool assembly that includes a hollow cutter having a bore. Additionally, the tissue stabilizer can have an internal helical groove (33, illustrated in FIG. 3B) that can be employed to guide the cutting tool assembly. The cutting tool assembly is receivable in the tissue stabilizer and can include a protrusion (31, illustrated in FIG. 2) that fits within the helical groove of the tissue stabilizer. The cutting tool assembly is movable relative to the tissue stabilizer to cause the cutter to advance while turning and excise material from the tissue sample with a synergistic effect of slicing and punching when the tissue sample is positioned below the opening of the support.

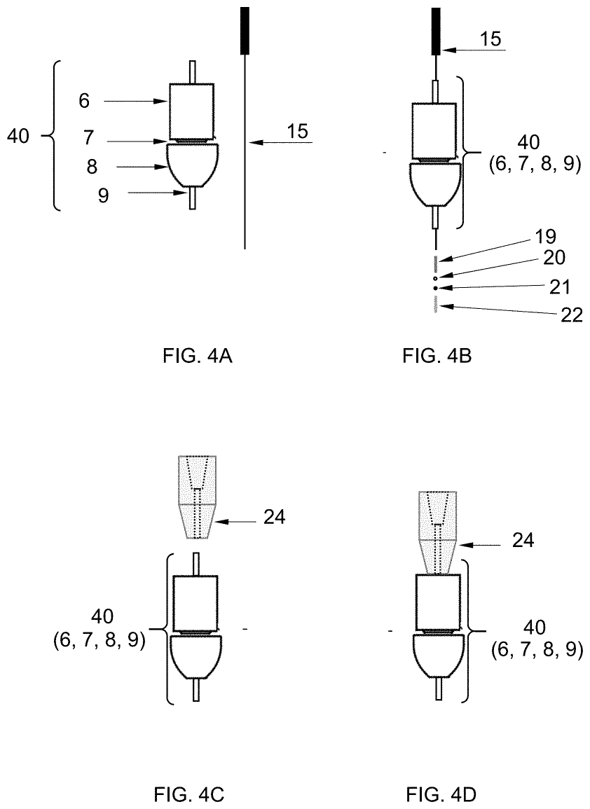

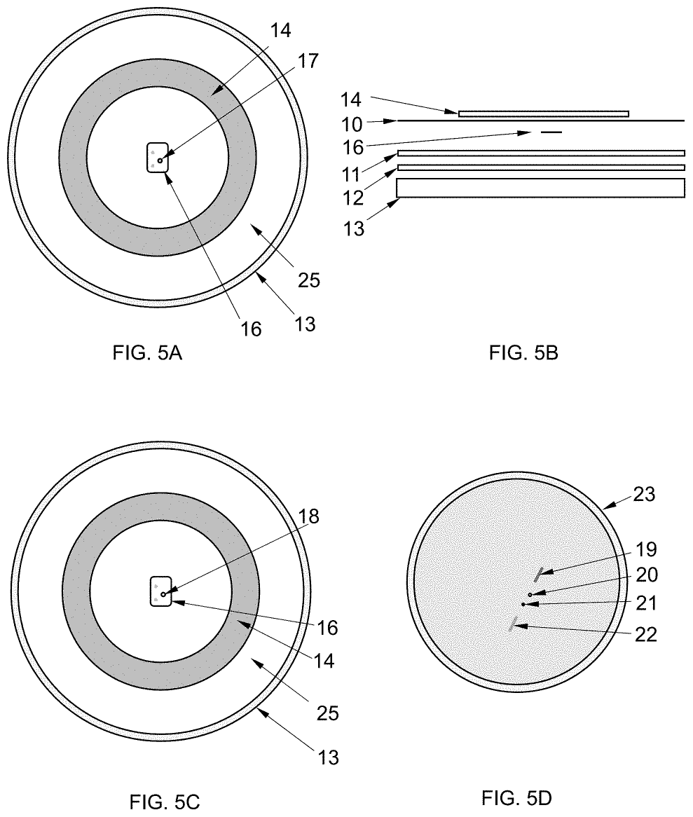

As shown in FIGS. 1 and 2, the device (100) can have a crescent-shaped base (1) and a support arm (2), which form the support of device. The base can be made of steel to provide stability to the tool and include a rubber underside that enhances the grip on the glass microscope stage. In the illustrated example, the support arm (2) is secured to the crescent shaped steel base (1) with screws (3). The crescent shaped steel base (1) and support arm (2) are carefully machined and the support arm (2) is secured with care so that they will be parallel to the microscope stage on which they rest. The tissue slice rests upon supporting layers that are also parallel to the microscope stage. The asymmetric opening in the support arm (2) is also machined with care and in such a way that the hole where the tissue stabilizer (4) enters is perpendicular to the base, the microscope stage, the supporting layers, and also the tissue slice. This ensures the accuracy of targeting and that when the cutter is inserted it punches perpendicular to the sample rather than at an angle. The tissue stabilizer (4) serves as a guide for the cutting tool assembly (components 5, 6, 7, 8, 9; see also FIGS. 4A-4D) and secures the tissue to the punching substrate (25) (comprising components 10, 11, 12; see also FIG. 5A) to allow removal of the cutter containing the punched tissue without adherence of surrounding tissue. The asymmetric tissue stabilizer (4) engages with the asymmetric hole in the support arm (2) to prevent rotation of the tissue stabilizer during the punching motion. A system for targeted material excision can include the device (100) and the punching substrate (25) that is configured for holding the tissue sample between layers of the substrate, as further described herein.

As shown in FIGS. 1 and 2, the tissue stabilizer (4) includes a body, e.g. a cylindrical body, that defines channel, e.g. a cylindrical channel with a helical groove, for receiving the cutting tool assembly (36) and guiding its motion via a protrusion on the chuck holder (5). A flange (34) extends outward from the stabilizer body, between an upper end of the stabilizer body and a lower end of the stabilizer body. The flange is ring shaped and has an outer diameter larger than the opening in the support arm. The flange is configured so as not to contact an upper surface of the support arm when a portion of the stabilizer is received in the opening of the support arm. This flange allows even pressure to be applied to the sample, regardless of the orientation of the tissue stabilizer in the support arm. Below this flange, there is an axially-oriented protruding external feature that engages with the asymmetric hole in the support arm (2), e.g., a linear ridge or rib (32) that is sized to fit a groove in the support arm (see FIG. 3A). A handle may be provided at the upper end of the stabilizer to allow easy insertion of the tissue stabilizer/cutting tool assembly into the support arm while within the confined space of the microscope stage. The tissue stabilizer (4) and the cutting tool assembly (36) can be separable parts or can form (e.g., can be coupled to form or can be combined into) one integrated module.

The cutter can be assembled by placing the collet (7) into the receiving sleeve (6) and then threading on the cap (8). Before completely tightening the cap (8), a needle-shaped cutter (9) is inserted such that it protrudes from the holder to the desired length from cap (8) of the part. Then the cap (8) is tightened securely. When the cap (8) is secured, the collet (7) (which is uniformly chamfered around its edge) makes contact with the inside of the cap (8) (which is also chamfered). This contact causes the four flexible ‘jaws’ of the collet (7) to be progressively pressed together, tightening their grip on the needle-shaped cutter (9) to hold it securely. The collet chuck (components 6, 7, 8) and cutter (9) are then secured in the chuck holder (5), completing the cutting tool assembly (36) (comprising components 5, 6, 7, 8, 9). The chuck holder (5) is designed to enhance the grip of the user to facilitate the punching motion. The cutting tool assembly (36) is guided through the tissue stabilizer (4) with its external protrusion engaging with the internal groove of the tissue stabilizer (4) to execute a combined turning and advancing motion and complete the tissue punching (see FIGS. 1 and 2).

FIGS. 4A-4D illustrate recovery of tissue from the hollow cutter of the device after the punching of live or fixed tissue. To recover the tissue, the chuck holder (5), collet chuck (6, 7, 8) and needle-shaped cutter (9) assembly are removed from the tissue stabilizer (4). FIG. 4A illustrates the collet chuck (6, 7, 8) and a needle-shaped cutter (9) assembly (40) after removal from the stabilizer, along with a plunger (15), which can be inserted into the bore of the needle-shaped cutter to recover tissue from the cutter. FIG. 4B illustrates the plunger (15) being inserted into the needle-shaped cutter during recovery of tissue from the collet chuck (6, 7, 8) and cutter (9) assembly (40). The plunger (15) contacts the grey silicone pre-load (19) which acts as a cushion to protect the excised tissue sample (21) that is sandwiched between one or more sheets of transparent film such as plastic film and/or cellophane (20) and the porous nutrient-containing under-pad (22). Cellophane is a transparent film made from cellulose that has low permeability to oxygen, moisture, oil, grease, and bacteria. In one example, the transparent film layer includes a layer of cellophane placed on top of the tissue and an over layer of plastic film (e.g., cling film), where the plastic film has a circular window of approximately the size of the tissue stabilizer's contact face through which the punches are made. This arrangement can reduce evaporation through the cellophane and avoid loss of tissue through adherence to the plastic film.

FIG. 4C illustrates a silicone adapter (24) that is designed to make a tight seal with both the cutter and a standard disposable pipet tip. When fitted to the cutter (9) as shown in FIG. 4D, the disposable tip of a pipet preloaded with culture medium can be pressed into the top of the adapter. When the medium is expelled, it rinses the internal bore of the cutter, ensuring that there is no carry-over of excised material between punches.

FIGS. 5A and 5B are top and exploded side views, respectively, illustrating tissue before punching. A tissue slice (16) supported by a porous, nutrient medium-containing under-pad (11) on a silicone support (12), which are placed in a petri dish (13). The tissue is covered by transparent film (10) which is secured with a stabilizer ring (14). The area of interest is indicated by a circle (17).

FIG. 5C is a top view of the tissue slice after punching. An area of removed tissue is indicated by a circle (18). FIG. 5D is a top view of material recovered from punch into culture plate with culture medium (23). In order of exit from the cutter, the recovered material includes: excised porous under-pad (22), excised targeted tissue region (21), excised transparent film (20), and excised grey silicone pre-load (19).

In a method of use for the micro punch device, the tissue to be punched is placed on a polyacrylamide under-pad (11) supported by a silicone cushion (12) in a plastic petri dish (13) and is covered with a sheet of cellophane (10) pre-wetted with culture medium (FIGS. 5A-5B). The cellophane sheet can be covered with piece of clear plastic film (not illustrated) with a central hole of a similar diameter to the contact face of the tissue stabilizer (4). The cellophane sheet and optional plastic film form a transparent layer that is secured with a stabilizing ring (14). The under-pad (11) and cushion (12) are translucent to allow illumination through the microscope stage. The elasticity of the silicone cushion (12) prevents dulling of the cutter (9) while providing a solid support for the polyacrylamide under-pad (11). The punching substrate (25) (comprising components 10, 11, 12), which now contains the tissue to be punched sandwiched between the under-pad (11) and the cellophane sheet (10), is placed within the crescent-shaped steel base (1) (see FIG. 1). The crescent shaped steel base (1) is designed to accommodate petri dishes (13) of different sizes and allow both rotation and lateral movement for ease of targeting. Placement of the petri dish (13) within the tool is via the open side of the crescent shaped steel base (1). After the petri dish (13) containing a tissue sample supported in the punching substrate (components 10, 11, 12) is placed into the steel metal base (1), the cutting tool assembly (components 5, 6, 7, 8, 9) is placed in the tissue stabilizer (4) and the tissue stabilizer (4) is inserted in the support arm (2) (see FIGS. 1 and 2).

Once the punch has been made, the tissue stabilizer (4) containing the cutting tool assembly (36) is removed and the punched tissue retrieved by using a plunger-like tool (15) that fits the inner bore of the cutter (9) to gently expel the tissue into plate containing culture medium (see FIGS. 4A-4B). Expelling punched tissue onto culture medium preserves the tissue viability. The inner bore of the cutter (9) can then be rinsed using a silicone adapter (24) and a standard pipettor fitted with a disposable pipet tip (see FIGS. 4C-4D). The adapter (24) is fitted onto the upper end of the needle-shaped cutter and a pipet tip pre-loaded with culture medium is press fit into the conical depression in the top of the adapter. The medium is then expelled from the pipettor, rinsing the inner bore of the cutter (9) and assuring that there is no carryover of excised material between punches.

The micro punch device can be made from various materials including but not limited to steel, brass, 3-D printed resin, and combinations thereof. In an example, the base (1) and the screws (3) are made from steel, the support arm (2) is made from aluminum, the tissue stabilizer (4) is made from brass and 3D printed resin, and the chuck holder (5) is 3D printed from a resin. In an example, the collet chuck (components 6, 7, 8) is made from brass and the cutter is made from stainless steel with a titanium nitride coating on the cutting edge. In an example, the punching substrate (25) includes plastic film, cellophane, polyacrylamide, and silicone, which are layered as illustrated in FIGS. 1, 2 and 5B. In general, the materials of the device and the materials of the substrate are chosen to be easily sterilizable.

Because minimizing the system size is important in making use of the small amount of space available at a microscope stage, the distance from the base to the support arm is minimized in order to fit under most dissecting microscopes.

A method to use this tool to efficiently excise, on a micron to millimeter scale, targeted areas of interest from a live tissue slice is as follows:
1. Place the crescent shaped steel base (1) of the micro punch device on the dissecting microscope's stage and place a petri dish (13) containing a tissue slice within the punching substrate (components 10, 11, 12) within the base (1) (FIGS. 1 and 2).
2. The rubber on the underside of the steel base (1) should be pressed firmly into the dissecting microscope's stage so that it grips/engages with the top of the stage
3. Secure the receiving sleeve (6) in the chuck holder (5), place the collet (7) into the receiving sleeve (6) and thread on the cap (8) of the collet chuck (components 6, 7, 8) (FIGS. 1 and 4A).
   a) Before securing the cap (8), insert the cutter (9) such that it protrudes from the cap (8) to the desired length.
   b) Securely tighten the cap (8).
4. Calibrate the micro punch device:
   a) Using the video camera on the microscope, position the micro punch device so that the hole in the support arm (2) is centered in the video monitor.
   b) Insert the tissue stabilizer/cutting tool assembly (4, 5, 6, 7, 8, 9) into the stabilizer (4) and make a initial punch into the punching substrate (components 10, 11, 12) (FIGS. 1 and 2).
   c) Remove the tissue stabilizer/cutting tool assembly (4, 5, 6, 7, 8, 9) and, using the camera software, make a fiducial mark on the image to indicate the location of the punched hole.
   d) Make a test punch on the punching substrate (components 10, 11, 12), using the fiducial mark on the video image to target an ink mark on the punching substrate. Remove the tissue stabilizer/cutting tool assembly (4, 5, 6, 7, 8, 9) and confirm that the cutter (9) is properly aligned. A snapshot of the location of the fiducial mark relative to the targeted ink can be compared to a snapshot of the location of the fiducial mark relative to the punched area to assess the accuracy of the targeting. Adjust the targeting as necessary by repositioning the fiducial mark on the video image to better reflect the location of the punch.
5. Place the live tissue on a polyacrylamide under-pad (11) on a silicone cushion (12) in a petri dish (13), place a pre-wetted cellophane sheet (10) over the live tissue and secure with the stabilizing ring (14) (FIGS. 5A-5B). All the components are kept properly hydrated by application of appropriate sterile nutrient medium.
6. Position punching substrate (components 10, 11, 12) containing the live tissue under the micro punch device and align the area of interest with the fiducial mark on the video monitor.
7. Punch a grey silicone sheet to pre-load the cutter (9) with an easily-visualized silicone core.
8. Insert the cutting tool assembly (components 5, 6, 7, 8, 9) into the tissue stabilizer (4) engaging the protrusion (31) of the chuck holder (5) in the internal helical groove (33) of the tissue stabilizer (4), align the external protruding rib (32) of the tissue stabilizer (4) in the groove of the opening (30) in the support arm (2), and guide the tissue stabilizer/cutting tool assembly (4, 5, 6, 7, 8, 9) into the support arm (2) until the tissue stabilizer (4) makes gentle contact with the cellophane sheet on top of the tissue. Take care that the cutter (9) does not contact the cellophane before the tissue stabilizer (4).
9. While maintaining gentle pressure with the tissue stabilizer (4), complete the tissue punching by turning the cutting tool assembly (components 5, 6, 7, 8, 9), allowing the helical motion that is generated as the protrusion of the chuck holder (5) moves within the helical groove of the tissue stabilizer (4) to guide the cutter into the sample and to execute a combined turning and advancing motion to couple the tangential and normal velocities of the cutting edge.
   a) The silicon substrate (12) provides flexibility to make sure that the tissue is cut and cushioned back into the cutter so that it is not lost. It also is transparent for illumination coming from the microscope
   b) The polyacrylamide under-pad and cellophane sheet keep the tissue moistened with culture medium and provide support for the tissue sample within the punching cutter and, along with the pre-loaded grey silicone core, also provide a means to verify complete recovery of the punched material from the bore of the cutter
10. Remove the tissue stabilizer (4) from the support arm (2) and remove cutting tool assembly (components 5, 6, 7, 8, 9) from the tissue stabilizer (4).
11. Use a plunger (15) to gently eject the tissue from the cutter into a culture plate (23) containing culture medium.
12. Attach the silicone adapter (24) to the top of the needle-shaped cutter and, using a pipettor fitted with a disposable tip pre-filled with nutrient medium, rinse the contents of the cutter bore into the culture plate (23) to ensure complete recovery of all the punched material (see, e.g., FIGS. 3D, 4D).
13. Additional areas of interest can be excised by repeating steps 5-12 as needed.

Punching regions of interest from live tissue with hand-held tools has been described in the literature (see, e.g., Jacobowitz, Brain Research, 80 (1974) 111-115; Johnston, et al., Biotechniques. 2018 Jan. 1; 64(1): 13-19.). Since hand holding the punch tool obstructs the view of the tissue slice on the microscope, it is difficult, if not impossible, to simultaneously visualize both the region of interest and the location of the punch in the absence of mechanical guidance for the punch. The punching stand of the present disclosure provides this guidance and the perpendicular hole in the support arm ensures the accuracy of punch targeting.

In addition, the previously described approaches either used thick (˜3 mm) tissue sections or they employed a mesh to hold down thinner (30-400 μm) sections for punching. Most live tissue sections are cut with a vibrating microtome and range from 150-300 μm. These tissues, including, for example, the ˜150 μm thick viable pancreas slices that are available to study islets, require the use of a hold-down. The use of a mesh hold-down limits the regions that are available for recovery. Testing on available tissue sections showed that it was very difficult, if not impossible, to position the mesh such that it did not obscure some of the islets of interest in the tissue sections, preventing their recovery. In addition, pancreas tissue has little mechanical stability and the mesh perforated the tissue when held with enough force to immobilize the sections. By replacing the mesh hold down with a cellophane sheet and holding the sheet in place with a tissue stabilizer that also acts as carrier and guide for the cutting tool assembly, improvements in targeted tissue excision can be achieved. The tissue stabilizer applies even pressure over a large area of the section, preventing pressure points that might cause unwanted perforation of the tissue. The cellophane sheet further prevents tissue damage, including adhesion to the tissue stabilizer.

In addition to the previously described approach of using a computer monitor for visualizing the microscopic view for punch calibration, the traditional microscope eyepiece can also be used for punch calibration by adding a marker, for example, cross-hairs, linear ticks, or a grid to the eyepiece. Alignment of the eyepiece marker with the center of the cutter by movement of the base (1) of the device completes the calibration.

Given that thick tissue sections are often fragile, sticky, and largely translucent, it is difficult to be certain that recovery of the punched tissue is complete. Addition of a pre-loaded grey silicone core in the cutter and an under-pad provides visible cores to bracket the excised tissue material and allow easy visualization of the excised material as it is recovered. The silicone cores also function as internal wipers within the bore of the cutter, to ensure complete recovery of the excised material from the cutter. The use of two different colors for the silicone cores allows the orientation of the recovered tissue to be determined.

The use of the silicone adapter (24) with a medium-filled pipet tip allows further rinsing of the bore of the cutter to ensure that there is no carryover of material between punches.

### EXEMPLIFICATION

### Example 1: Mouse Pancreas

A prototype of the punching tool was used to excise tissue from a mouse pancreas. The punching tool allowed the user to consistently punch the tissue and expel the excision into another petri dish for further study. A nylon mesh was used to immobilize the tissue on a silicone under-pad.

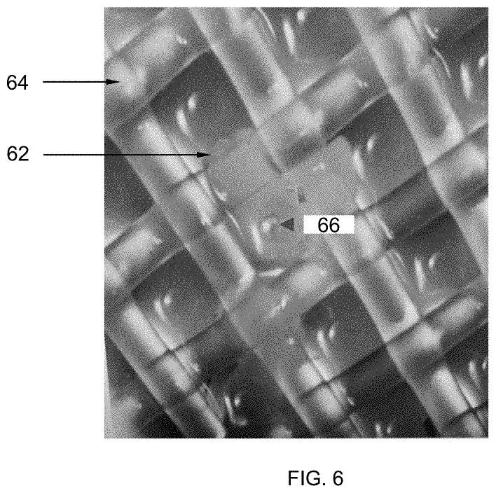

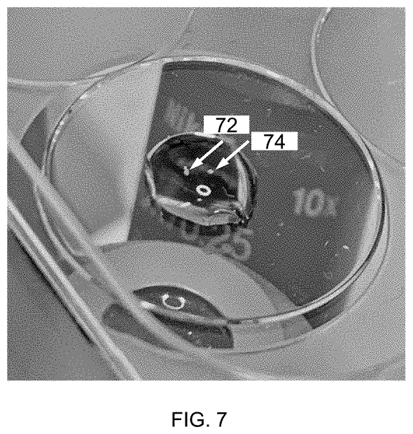

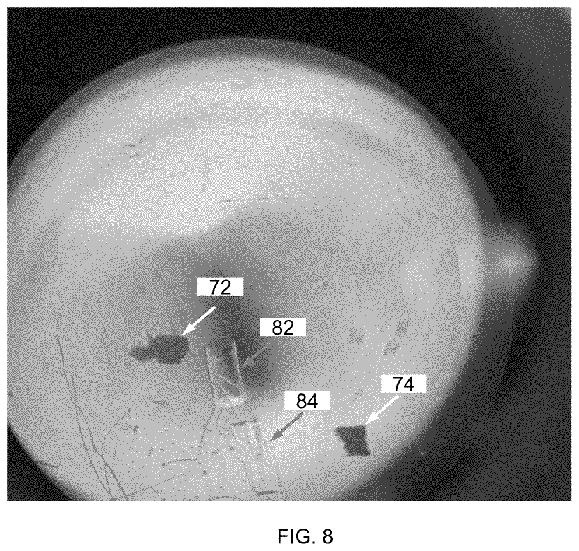

FIG. 6 shows a slice of live mouse pancreatic tissue (62) immobilized on the silicone under-pad by nylon mesh (64). In the center of the live tissue sample, there is a circular empty excision (66) that was made by the tool (indicated by the red arrowhead). This example illustrates that the tool can be used to punch all the way through the tissue, which is evidenced by the translucent silicon substrate that is visible through the punched hole. In addition, it was demonstrated that the tool can be used to retrieve the punched tissue (72, 74) and place it onto the culture media that preserves cell viability, as seen in FIG. 7. The live tissue sample, along with a second tissue sample from another successful punch, was then moved to another microscope, as can be seen in FIG. 8. This allowed the silicone under-pad cores (82, 84) to be visualized along with the excised tissue samples (72, 74).

### Example 2: Human Pancreas

The example methods and results described below are based on an abstract and poster presentation (Sambra D. Redick, Katerina Angjeli, David M. Blodgett, Alan Derr, Sally C. Kent, Yihao Zheng, David M. Harlan, Development of an islet “micropunch” to isolate and characterize single islets from donors with and without T1D through the nPOD pancreatic slice program. Network of Pancreatic Organ Donors with Diabetes (nPOD) 13^{th} Annual Meeting. Virtual, Feb. 22-24, 2021).

Human islets are known to be very heterogeneous, even within a single donor's pancreas. Not only does the size and cellular constituency (α-, δ-, γ-, and β-cell proportion) vary from islet to islet within any individual human pancreas, but also in donors with T1D, some islets may be infiltrated by inflammatory cells, while other islets in the same donor pancreas may appear to be uninflamed. Since islet inflammation may influence islet function, endocrine cell numbers, and individual islet cell gene expression, it is useful to develop assays that assess those variables from a single human donor. The successful development of such techniques can provide data on questions like:
1. How does an individual islet's cell constituents influence that islet's insulin and glucagon secretion in response to physiological stimuli?
2. Do β-cells (or a-cells) from islets with infiltrating T cells display unique gene expression patterns relative to islets from the same donor without demonstrable insulitis?
3. Are the functional responses of inflamed islets demonstrably different from those of uninflamed islets of similar size and cellular makeup?

Data addressing these knowledge gaps may shed new light on the basic pathophysiological processes underlying T1D, and therefore identify potential therapeutic targets.

Methods

A custom micropunch tool was developed that allows sampling of individual islets and surrounding tissue from viable pancreas slices, using biopsy needles of 350, 560, or 860 μm bore to accommodate islets of various sizes. The punched islets from donors with or without T1D are recovered in a well of an HTS Transwell® plate. After overnight culture to recover from isolation shock, the transwell is acclimated in basal glucose Krebs-Ringer Bicarbonate+HEPES (KRBH) solution for 1 hour. The transwell insert is then serially passed into custom-printed low volume reservoir plates containing basal glucose KRBH, high glucose KRBH, low glucose KRBH+epinephrine, and KCl. Incubation time in each buffer is 15 minutes. The insert is gently centrifuged after each step to minimize carry over and maximize recovery of solutions. These culture supernatants are then assayed for insulin and glucagon content using commercially available ELISA kits. The same islets studied for their functional responses are then dissociated into single cells, and the islet's cellular constituents and each individual cell's transcriptome is determined using techniques well-established in our laboratory.

Summary of Results

Techniques were developed and data obtained showing:
1. A micropunch tool that can isolate single, viable islets and peri-islet T cells from pancreas slices
2. Static assays for determining the function of individual islets, i.e. insulin and glucagon secretion in low and high glucose concentrations.
3. The transcriptome of single cells from individual islets has been developed for traditionally isolated islets and is now being adapted for micropunch-isolated islets.

These techniques may be applied to a single islet, revealing that islet's secretory functionality, cellular constituents, and each cell's gene expression profile.

Conclusions

Many of the remaining fundamental knowledge gaps in the understanding of T1D pathophysiology relate to the patchy “vitiligo-like” nature of the disease, e.g., why are some islets inflamed while others are not, and what impact do infiltrating or peri-islet T cells have on an islet's function, cellular constituents, and individual cell's gene expression. The techniques developed can provide a platform for gaining insight into such issues.

### Example 3 Human Pancreas

A prototype punching tool was used to excise tissue from a human pancreas slice. The punching tool allowed the user to consistently punch the tissue and expel the excision into another petri dish for further study. The punching sandwich included a cellophane overlay, the tissue slice, an acrylamide under-pad, and a silicone under-pad on a silicone support.

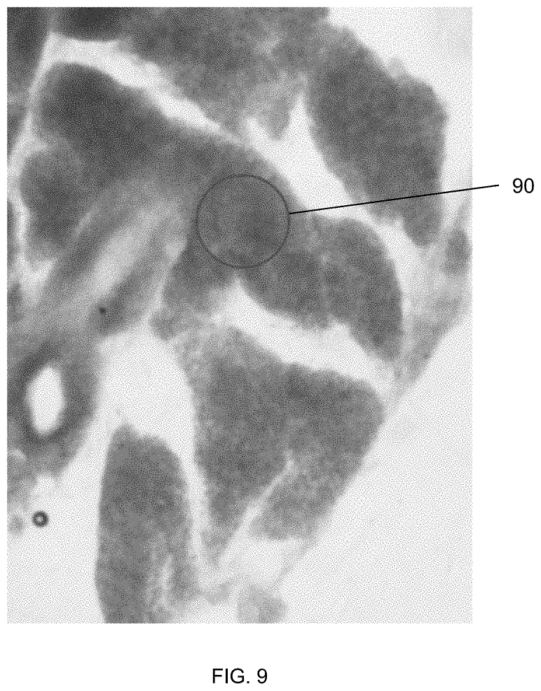

FIG. 9 shows a region of the pancreas slice prior to punching. The red circle (90) indicates the islet targeted for excision.

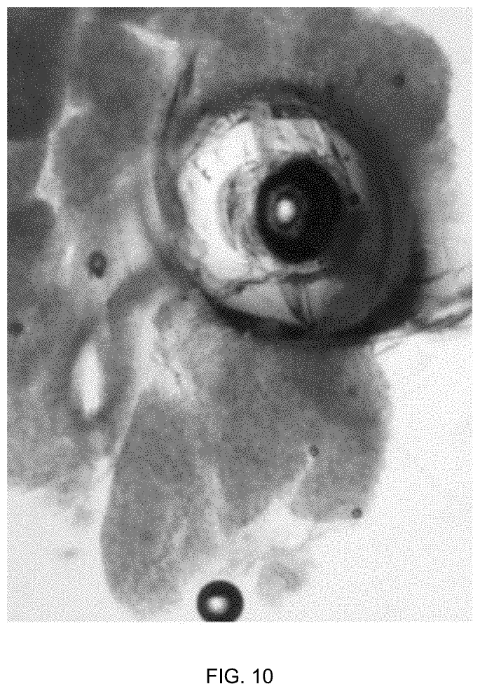

FIG. 10 shows the same region of the pancreas slice following punching with the punching tool. The targeted area is now within a clear region resulting from tissue removal. Tissue excision resulted in slight distortion of the tissue and the introduction of a small air bubble at the bottom of the image.

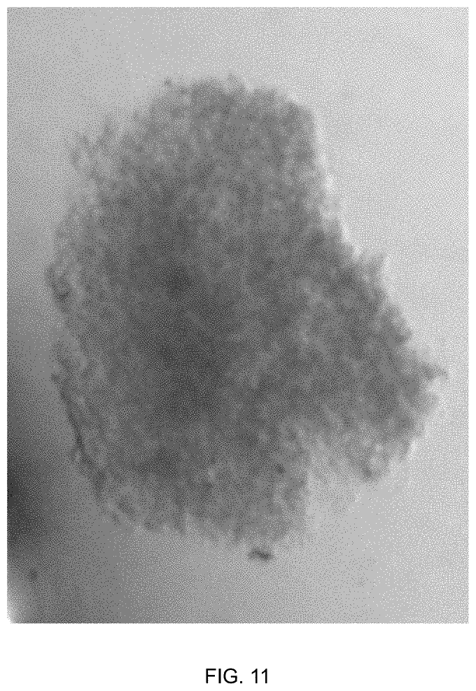

FIG. 11 shows a high magnification view of the tissue recovered from the hollow needle of the punching tool.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A device for targeted material excision from a tissue sample, the device comprising:
a support defining an opening and providing a region below the opening for positioning a tissue sample;
a tissue stabilizer receivable in the opening of the support, the tissue stabilizer defining a shape that complements a shape of the support defining the opening; and
a cutting tool assembly receivable in the tissue stabilizer, the cutting tool assembly including a hollow cutter having a bore, the cutting tool assembly being movable relative to the tissue stabilizer to cause the cutter to excise material from the tissue sample and retain the excised material within the bore when the tissue sample is positioned below the opening of the support.

2. The device of claim 1, wherein the cutting tool assembly includes a collet chuck configured to hold the hollow cutter in an orientation with an excise end of the cutter directed toward the tissue sample, and further includes a chuck holder to couple to the collect chuck to facilitate manipulation of the cutting tool assembly by a user.

3. The device of claim 1, wherein the support includes a base and a support arm attached to the base and defining the opening of the support.

4. The device of claim 3, wherein the base is crescent shaped and configured to at least partially surround the tissue sample when the tissue sample is positioned below the opening of the support.

5. The device of claim 1, further comprising a plunger receivable in the bore of the hollow cutter to expel material excised from the tissue sample.

6. The device of claim 1, wherein the opening in the support is asymmetric and wherein the shape defined by the tissue stabilizer complements the asymmetric opening.

7. The device of claim 1, wherein the tissue stabilizer includes an internal helical groove, the cutting tool assembly includes on its outer surface a protrusion, and wherein movement of the cutting tool assembly relative to the tissue stabilizer is guided by the protrusion traversing the internal helical groove.

8. The device of claim 1, wherein the tissue stabilizer and the cutting tool assembly are separable parts or wherein the tissue stabilizer and the cutting tool assembly form one integrated module.

9. The device of claim 1 in combination with a substrate, wherein the substrate enables holding of the tissue sample between layers of the substrate.

10. The device and substrate combination of claim 9, wherein the layers of the substrate include a sheet of transparent film, a porous under-pad, and a silicone cushion.

11. A method for targeted excision of material from a tissue sample, the method comprising:
positioning a tissue sample below an opening in a support;
inserting a cutting tool assembly into a tissue stabilizer, the cutting tool assembly including a hollow cutter;
aligning the tissue stabilizer and cutting tool assembly with the opening in the support and guiding the tissue stabilizer and cutting tool assembly through the support; and
while stabilizing the tissue sample, moving the cutting tool assembly relative to the tissue stabilizer to cause the hollow cutter to excise material from the tissue sample.

12. The method of claim 11, further comprising holding the tissue sample between layers of a substrate including a sheet of transparent film, a porous under-pad, and a silicone cushion.

13. The method of claim 12, wherein stabilizing the tissue sample includes contacting the substrate with the tissue stabilizer.

14. The method of claim 13, wherein stabilizing the tissue sample includes securing the tissue sample between the sheet of transparent film and the porous under-pad with a stabilizing ring.

15. The method of claim 11, further comprising inserting a plunger into a bore of the hollow cutter to expel material excised from the tissue sample.

16. The method of claim 15, further comprising removing the cutting tool assembly from the tissue stabilizer prior to inserting the plunger into the bore of the hollow cutter.

17. The method of claim 11, wherein the opening in the support is asymmetric and wherein the shape defined by the tissue stabilizer complements the asymmetric opening.

18. The method of claim 11, wherein the tissue stabilizer includes an internal helical groove, the cutting tool assembly includes on its outer surface a protrusion, and wherein movement of the cutting tool assembly relative to the tissue stabilizer is guided by the protrusion traversing the internal helical groove, the movement causing the cutter to excise material from the tissue sample with a synergistic effect of slicing and punching.

19. The method of claim 11, wherein moving the cutting tool assembly relative to the tissue stabilizer includes turning and advancing the cutting tool assembly relative to the tissue stabilizer and the tissue sample.

20. A system for targeted material excision from a tissue sample, the system comprising:
a substrate for holding a tissue sample between layers of the substrate; and
a device for targeted material excision, the device including:
a support defining an opening and providing a region below the opening for positioning the tissue sample held by the substrate;
a tissue stabilizer receivable in the opening of the support, the tissue stabilizer defining a shape that complements a shape of the support defining the opening; and
a cutting tool assembly receivable in the tissue stabilizer, the cutting tool assembly including a hollow cutter having a bore, the cutting tool assembly being movable relative to the tissue stabilizer to cause the cutter to excise material from the tissue sample and retain the excised material within the bore when the tissue sample is positioned below the opening of the support.
